(19) United States
(12) Patent Application Publication
(10) Pub. No.: US 2004/0242724 A1
(43) Pub. Date: Dec. 2, 2004

(54) Acid decomposable compound

(76) Inventor: Yutaka Saito, Sakura (JP)

Correspondence Address: Yutaka Saito, 1639-3 Ino cho, Sakura, Chiba, JP

(21) Appl. No.: 10/752,975

(22) Filed: Jan. 5, 2004

(30) Foreign Application Priority Data
Jun. 1, 2003 (JP) 2003-000181

(51) Int. Cl. C07C 69/52; C07D 207/444

(52) U.S. Cl. 523/125; 548/548; 560/224

(57) ABSTRACT

An acid decomposable compound of the present invention is a compound having acid decomposable groups and thermally-bond-formable groups in one molecule in a certain order. The acid decomposable compound contains at least a core structure, acid decomposable groups which are binding to the core structure and thermally-bond-formable groups which are binding to the acid decomposable groups, wherein the thermally-bond-formable groups undergo Diels-Alder Reactions, metathesis reactions or other thermal-addition reactions.

### BACKGROUND OF THE INVENTION

This invention relates to new decomposable compounds. The new decomposable compounds are useful as decomposable plastics, decomposable plastic resins, photosensitive resins, photoresists or the like. The example of the compounds will involve decomposable plastics, photoresist materials, inks, paints, lenses and photodiscs.

Disposal of synthetic resins such as plastics puts a lot of strain on the environments. That is why a lot of researches in which degradability or recyclable ability was imparted to synthetic resins have been conducted.

Some type of Diels-Alder Reactions have been known as reversible reactions. A polymer synthesized through the reversible Diels-Alder Reaction undergoes a retro-Deals-Alder Reaction by heating, causing a decomposition or bond exchanging reaction in its molecule. A heat crosslinkable polymer composition containing a polymer having a furan moiety in its side chain and low molecular weight molecule having maleimide structure has been reported in Macromolecules, 31, p314-321 (1998). Although Diels-Alder and retro-Deals-Alder Reaction proceeded in the polymer composition when it was heated, the polymer kept its high molecular weight so the composition did not achive decomposability. A mixture of a low molecular weight compound having plural furans in the molecule and another low molecular weight compound having plural maleimides in the molecule has been reported in Science, 295, p1698-1702 (2002) by Chen et al. The thermal treatment of the mixture caused a polymerization reaction, forming a network polymer capable of undergoing a crosslinking or decomposition reaction. The resultant polymer provided repairability and was useful as a structural material. However, it is possible that the cost for disposal of the resultant polymer after it is used is high because a temperature at which the network polymer can undergo a decomposition reaction must be high. In addition, no suggestion for application of the polymer as a functional material such as a photoreactive polymer was offered in the report.

The inventor who has invented the present invention got the idea that it is possible to obtain a new polymer available as a functional material such as a photoresist while having decomposability by using a thermal crosslinking reaction such as a Diels-Alder Reaction. Hereafter, prior arts of photoresists will be explained.

Chemically amplified resists have been widely used for the production of integrated circuits. A chemically amplified resist generally contains a polymer which acts as a matrix, and a photo acid generator which can generate an acid in response to incident light. The polymer is mainly composed of a base-soluble resin in which all or some groups imparting alkali solubility to it are protected with acid decomposable groups.

Next, a photoreaction mechanism of the photoresist composed of the polymer will be described. When the resist on a substrate is irradiated with light, acids are generated from the photo acid generator, eliminating the acid decomposable groups, making the polymer alkali-soluble. Then the resist is subjected to alkali development, leaving non-irradiated parts of the resist on the substrate.

In the case when conventional photoresists are used, however, only the solubility in a light-irradiated region is increased; but that in a non-light-irradiated region remains unchanged, leaving room for improving the contrast between the regions.

### BRIEF SUMMARY OF THE INVENTION

The first object of the present invention is to provide a material which is easily disposable and puts low strain on the environment after its disposal while having reparability.

The second object of the present invention is to provide a new compound capable of being used as photosensitive resin.

In order to achieve the aforementioned objects, the acid decomposable compound of the present invention is a compound having acid decomposable groups and thermally-bond-formable groups in one molecule in a certain order.

### BRIEF DESCRIPTION OF THE DRAWINGS

FIG. 1 shows an NMR spectra chart of 1-(2-furyl)ethylmethacrylate.

FIG. 2 shows an NMR spectra chart of poly(1-(2-furyl)ethylmethacrylate).

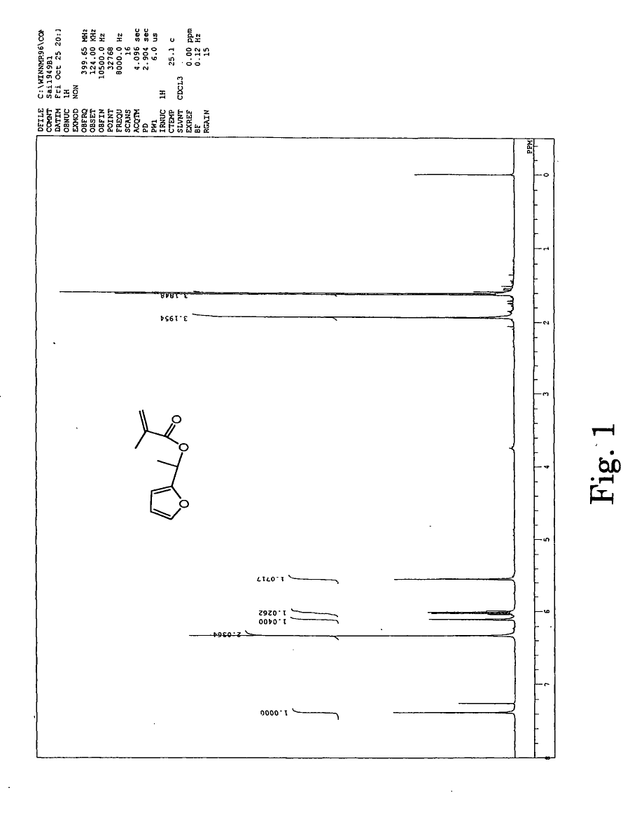

FIG. 1 shows an NMR spectra chart of 1,3,5-tris(methylurfuryloxycarbonyl)benzene.

### DETAILED DESCRIPTION OF THE INVENTION

The first embodiment of the present invention is an acid decomposable compound containing at least a core structure, acid decomposable groups which are binding to the core structure and thermally-bond-formable groups which are binding to the acid decomposable groups as shown in following Formula (1):

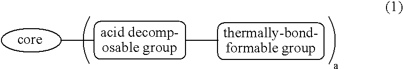

wherein the core structure represents a carbon atom, a silicon atom, an oxygen atom, a sulfur atom, a boron atom and an atom of which electron negativity difference to carbon is 2.0 or less or an organic group; in addition, the core may make up all or part of the acid decomposable groups; and the acid decomposable groups represents organic groups containing at least one of groups selected from the following groups that: tertiary ester bonds, secondary arylester bonds, tertiary oxycarbonyloxy bonds, thioester bonds, acetal bonds, tertiary ether bonds, secondary arylester bonds, tertiary thioether bonds, secondary thioether bonds and these bonds partially substituted with electron donating groups; in addition, the acid decomposable groups may make up all or part of the thermally-bond-formable groups; and the thermally-bond-formable groups represents organic groups which can undergo Diels-Alder Reactions, metathesis reactions or other thermal-addition reactions; where “a” represents positive integer, and when “a” is two or more, acid decomposable groups may be different from each other and thermally-bond-formable groups may be different from each other.

Organic groups for the core structure can be any kind of organic groups, and preferably a multivalent (preferably more than three) structure such as structure based on tri-substituted benzenes, on dendrimers or oligomers with branched structure or side chains available for having an acid decomposable structure, or on polymers with branched structure or side chains available for having an acid decomposable structure. The core preferably has a poly-branched structure, and more preferably tri- or more branched structure.

Examples of the poly-branched structure include: 1,3,5-tri-branched benzene derivatives, 3,5,3′,5′-tetra-branched derivatives, 1,3,5-triphenylbenzene derivatives, a poly-branched cycloalkyl derivatives which may or may not have bridging carbon in its molecule and having 4 to 12 carbons, a poly-branched adamantanes, and tri-branched tertiary amine. A poly-branched structure in a compound imparts higher solubility or amorphous properties to the compound.

Preferable examples of the poly-branched structures are shown in the following Formula (29) to (38).

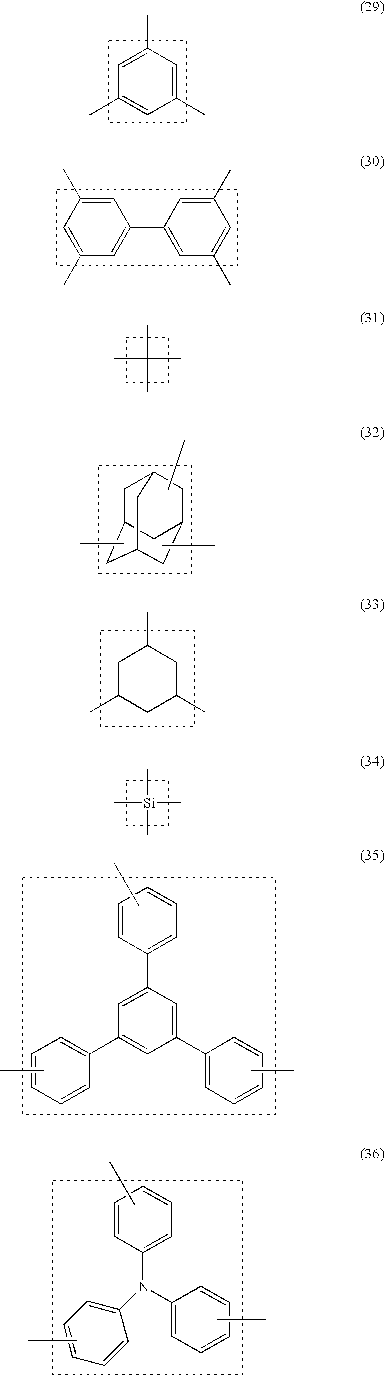

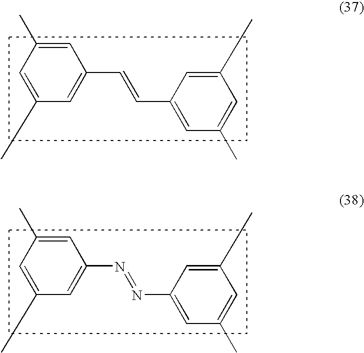

Other preferable examples of core structures are oligomer structres or polymer structures mainly made up from monomers such as acrylic acid derivatives, methacrylic acid derivatives, hydroxystyrene derivatives, norbornene derivatives, maleinic acid derivatives, and maleimide derivatives. The other examples of oligomers and polymers include polyesters, polyolefines, and the other polymer structures not be exemplified here also can be used for the core structure of the present invention.

In the present invention, the term “acid decomposable group” means atom groups which are capable of being decomposed by an acid with pKa of 10 or less below 200° C., or preferably below 150° C.

The typical example of the acid decomposable group is a functional group protected with an acid decomposable group.

In the present invention, the acid decomposable group may include a part of a thermally-bond-formable group or a whole thermally-bond-formable group.

Examples of the acid decomposable groups include tertiary ester bonds, secondary arylester bonds, tertiary oxycarbonyloxy bonds, thioester bonds, acetal bonds, tertiary ether bonds, secondary arylester bonds, tertiary thioether bonds, secondary thioether bonds and these said bonds partially substituted with electron donating groups.

To explain more in detail, preferable structures of the acid decomposable groups (atom groups) will be exemplified in following Formula (2) to (8), however, no limitation is imposed by the exemplified structures.

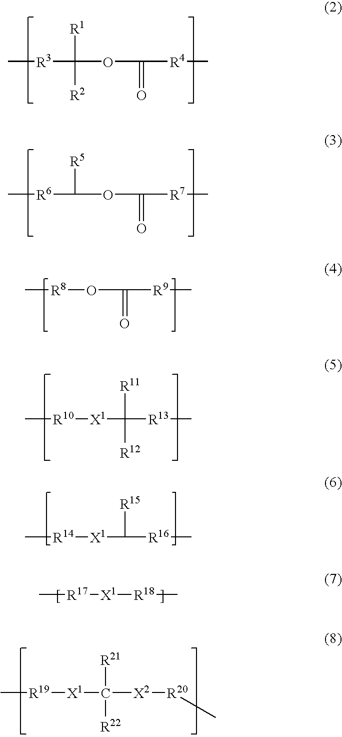

In Formula (2) to (8), R1, R2, R5, R11, R12, R15, R21 and R22, respectively, represent a linear, branched or cyclic alkyl group having 1 to 6 carbons which may have substituent groups; an aryl group having 5 to 10 carbons which may have substituent groups; an alkoxy group which may have substituent groups; a thioalkoxy group which may have substituent groups; a vinyl group which may have substituent groups; a saturated or unsaturated heterocyclic group which may have substituent groups; and a silyl group which may have substituent groups.

Specifically, R1, R2, R5, R11, R12, R15, R21 and R22, respectively, represent a hydrocarbon group such as methyl group, ethyl group, n-propyl group, 1-methylethyl group, cyclopropyl group, n-butyl group, 1-methylpropyl group, 2-methylpropyl group, cyclobutyl group, n-pentyl group, pentyl group with branched structure, cyclopentyl group, n-hexyl group, hexyl group with branched structure, cyclohexyl group, aryl groups and these hydrocarbon groups having substituent groups such as alkyl groups, alkoxy groups, enter groups, nitro groups, halogens, cyano groups, hydroxy groups, thiol groups; or ether groups such as methyloxy group, ethyloxy group, n-propyloxy group, 1-methylethyloxy group, cyclopropyloxy group, n-butyloxy group, 1-methylpropyloxy group, 2-methylpropyloxy group, cyclobutyloxy group, n-pentyloxy group, pentyloxy group with branched structure, cyclopentyloxy group, n-hexyloxy group, hexyloxy group with branched structure, cyclohexyloxy group, aryloxy groups and these ether groups having substituent groups such as alkyl groups, alkoxy groups, enter groups, nitro groups, halogens, cyano groups, hydroxy groups, thiol groups; or aryl groups such as phenyl group, furanyl group, thionyl group, pyridyl group, naphtyl group and these aryl groups having substituent groups such as alkyl groups, alkoxy groups, enter groups, nitro groups, halogens, cyano groups, hydroxy groups, thiol groups, or aryl groups; or silyl groups such alkyl silyl group, dialkyl silyl group, silyl group substituted with aryl group.

R3, R4, R7, R9, R13, R19 and R20, respectively, represent a bivalent organic group, a silyrene group, an oxygen atom, a sulfur atom, a secondary or tertiary boron atom or a secondary or tertiary nitrogen atom and any combination of these exemplified groups or atoms wherein the organic group is a group selected from an alkyrene group which may have substituent groups a vinylene group which may have substituent groups, a carbonyl group, an arylene group which may have substituent groups.

In addition, in Formula (2), R1, R2, R3 and an R3 binding group (not shown, it is a part of the core structure or thermally-bond-formable group) may form a cyclic bonding structure between any of them; R4 and an R4 binding group (not shown, it is a part of the core or thermally-bond-formable group) may form a cyclic bonding structure between them.

In Formula (3) and (6), R6 and R16, respectively, represent an organic group which containing at least the following group at the closer side to the acid dissociative site of it: an arylene group which may have substituent groups, a vinylene group which may have substituent groups, a methylene group which have electron donating groups, a vinyleneoxy group which may have substituent groups, or a silyrene group which may have substituent groups.

The other examples of R6 and R16 include a vinylene group having a conjugated multiple bond; and a methylene group or a methin group having electron donating group such as methoxymethylene group, methylthiomethylene group, methoxyethoxymethylene group and 2-(trimethylsilyl)ethoxymethylene group, 2-tetrahydropyranylene group; a furanylene group with an electron donating group; thionylene groups with an electron donating group; a phenylene group with an electron donating group; a vinylene groups with an electron donating group; an aryloxymethylene group, which may have a substituent group, such as benzyloxymethylene groups, furanyloxymethylene groups and thionyloxymethylene groups; a vinyloxylene group which may have a substituent group; an N-phthalimide methylene group which may have a substituent group; a 9-anthrylmethylene group which may have a substituent group; and a silylene group such as trimethisilylene group, triethylsilylene group and phenyldimethylsilylene group.

R6 or R16 is preferably an arylene group such as a phenylene group, a naphtylene group, an anthranylene group, a furanylene group, a pyridilene group, and a thiophenylene group; a phenyleneoxy group, an arylenevinylene group such as a phenylenevinylene group, a furanylenevinylene group and a pyridylenevinylene group, respectively; and more preferably a furanylene group, a furanyloxylene group or a furanylenevinylene group.

In addition, in Formula (3), R5, R6 and an R6 binding group (not shown, it is a part of the core or thermally-bond-formable group) may form a cyclic bonding structure between any of them; R7 and an R7 binding group (not shown, it is a part of the core or thermally-bond-formable group) may form a cyclic bonding structure between them.

In Formula (4) and (7), R8 and R18 respectively represent an organic group which containing at least the following group at the closer side to the acid dissociative site of it: a bivalent organic group selected from a tetrahydropiranylene; an arylene having electron donating group; a vinylene group having electron donating groups; an aryloxymethylene group which may have substituent groups such as a furanyloxymethylene group and a thionyloxymethylene group; an N-phthalimide methylene group which may have substituent groups; a 9-anthryl methylene group which may have substituent groups; and silylene groups which may have substituent groups such as a trimethyl silylene group, a triethyl silylene group and a phenyl dimethyl silylene group.

In addition, in Formula (4), R8 and an R8 binding group (not shown, it is a part of the core or thermally-bond-formable group) may form a cyclic bonding structure between them, R9 and an R9 binding group (not shown, it is a part of the core or thermally-bond-formable group) may form a cyclic bonding structure between them, in Formula (5) to (8), X1 and X2 respectively represent oxygen atom or sulfur group.

In Formula (5) to (7), R10, R14 and R17 respectively represent an organic group which containing at least the following group at the closer side to an acid dissociative site of it: a bivalent arylene group which may have substituent groups and a bivalent vinylene group which conjugates to an aryl group and may have substituent groups.

Also in Formula (5), R11, R12, R13 and an R13 binding group (not shown, it is a part of the core or thermally-bond-formable group) may form a cyclic bonding structure between any of them; R10 and an R10 binding group (not shown, it is a part of the core or thermally-bond-formable group) may form a cyclic bonding structure between them.

Also in Formula (6), R15, R16 and an R16 binding group (not shown, it is a part of the core or thermally-bond-formable group) may form a cyclic bonding structure between any of them, R14 and an R14 binding group (not shown, it is a part of the core or thermally-bond-formable group) may form a cyclic bonding structure between them.

Also in Formula (7), R17 and an R17 binding group (not shown, it is a part of the core or thermally-bond-formable group) may form a cyclic bonding structure between them, R18 and an R18 binding group (not shown, it is a part of the core or thermally-bond-formable group) may form a cyclic bonding structure between them.

Also in Formula (8), R21, R22, R19 and an R19 binding group (not shown, it is a part of the core or thermally-bond-formable group) may form a cyclic bonding structure between any of them, R21, R22, R20 and an R20 binding group (not shown, it is a part of the core or thermally-bond-formable group) may form a cyclic bonding structure between any of them, but when R21 forms a cyclic bonding structure with R19 or the R19 binding group, R21 or R22 does not form a cyclic bonding structure with R20 or the R20 binding group; when R21 forms a cyclic bonding structure with R20 or the R20 binding group, R21 or R22 does not form a cyclic bonding structure with R19 or the R19 binding group; when R22 forms a cyclic bonding structure with R19 or the R19 binding group, R22 does not form a cyclic bonding structure with R20 or the R20 binding group.

All of the above mentioned acid decomposable groups may be whole or a part of the thermally-bond-formable groups which will be explained in detail later.

The thermally-bond-formable group is an atom group which has low reaction rate under a condition below 0° C. but is increased practically in their reactivity under a condition between 0° C. and 300° C., or preferably between 5° C. and 200° C. so as to form covalent bonds. That is, the thermally-bond-formable groups include atom groups which can undergo covalent-bond-forming reactions by heating with a heating apparatus or by leaving them under a certain temperature.

The thermal-covalent-bond-forming reactions may or may not contain catalysts, as required. Also the thermal-covalent-bond-forming reactions may include multi-step reactions after which new covalent bonds are formed, for example, successive reactions consisting of a retro-Diels-Alder Reaction and a Diels-Alder Reaction in which bond decomposition and new bond formation occur successively.

It is required to not dissociate the acid decomposable groups in the course of thermal-covalent-bond-forming reactions. In other words, a lot of atom groups are available for the thermally-bond-formable groups insofar as they undergo a thermal-bond-forming reaction without degrading the acid decomposable groups.

Examples of thermal-covalent-bond-forming reactions include Diels-Alder Reactions, en reactions, metathesis reactions and reactions catalyzed with a base catalyst. Specifically, Diels-Alder Reactions between furans and maleimides, Diels-Alder Reactions between anthracenes and maleimides, and metathesis reactions catalyzed by the Grabbs catalyst or the like are preferable examples. Diels-Alder Reactions usually need no metal catalyst so that such kind of reactions are preferably applied for a thermal-covalent-bond-forming reactions of the present invention, when applying for a photoresist where extremely low metal contamination is required. Of these reactions, reversible reactions are preferable. In the case where the reversible reactions are applied for a thermal-covalent-bond-forming reaction in the present invention, many advantageous effects will be obtained. For example, recycling property and self-mending property by heating will be obtained, specifically, when a composition containing the compound which undergoes reversible thermal-covalent-bond-forming reactions is used as a photoresist and irradiated with light, a degree of edge-line-roughness between a light irradiated region and non-light irradiated region will decrease by heat annealing. These examples explained above do not impose any limitation.

Examples of preferable structures for atom groups capable of undergoing Diels-Alder Reactions include a furan ring structure, a maleimide ring structure, a diene structure specifically a conjugated diene structure, a double bond structure specifically a double bond having electron-donating groups, an anthracene structure and fullerene structures. One of examples of preferable structures for atom groups capable of undergoing ene reaction is a double bond. One of examples of preferable structures for atom groups capable of undergoing metathesis reaction is also a double bond. Some radical reactions including a covalent-bond-formation by a heat treatment such as a radical reaction between a stable radical and a compound with a benzylic hydrogen are also available.

To explain further, preferable examples of structures for thermal-bond-forming groups will be shown in the following formula (23) to (28), and it is noted that these examples do not impose any limitations to the present invention.

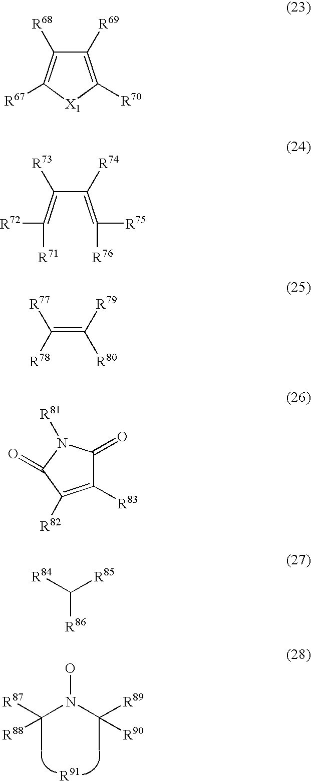

In Formula (23) and (27), R67-R84, respectively, represent a hydrogen atom; a halogen atom; a hydroxy group; a thiol group; a cyano group; a nitro group; an amino group; an alkylamino group; a dialkylamino group; a linear, branched or cyclic alkyl group having 1 to 6 carbons which may have substituent groups; an aryl group having 5 to 10 carbons which may have substituent groups; an alkoxy group which may have substituent groups; a thioalkoxy group which may have substituent groups; a vinyl group which may have substituent groups; a saturated or unsaturated heterocyclic group which may have substituent groups; and a silyl group which may have substituent groups.

Specifically, R67-R84, respectively, represent a hydrogen atom, a halogen atom, a hydroxy group, a thiol group, a cyano group, a nitro group, a methyl group, an ethyl group, an n-propyl group, a 1-methylethyl group, an n-butyl group, a 1-methylprooyl group, a 2-methylpropyl group, a cyclobutyl group, an n-pentyl group, a pentyl group with a branched structure, an n-hexyl group, a hexyl group with a branched structure, a cyclohexyl group, an alkyl group containing 7 to 20 carbons having a linear or branched structure, an allyl group; these said alkyl groups having substituent groups such as alkyl groups, alkoxy groups, ester groups, nitro groups, halogen atoms, cyano groups, hydroxy groups and thiol groups; a methyloxy group, an ethyloxy group, an n-propyloxy group, a 1-methylethyloxy group, a cyclopropyloxy group, an n-butyloxy group, a 1-methylpropyloxy group, a 2-methylpropyloxy group, a cyclobutyloxy group, an n-pentyloxy group, a pentyloxy group with a branched structure, a cyclopentyloxy group, an n-hexyl group, a hexyloxy group with a branched structure, a cyclohexyloxy group, an alkyloxy group having 7 to 20 carbon atoms with a linear or branched structure; these said alkoxy groups having substituent groups such as alkyl groups, alkoxy groups, ester groups, nitro groups, halogen atoms, cyano groups, hydroxy groups and thiol groups; aryl groups such as a phenyl group, a furanyl group, a thionyl group, a pyridyl group, a naphtyl group; these said aryl groups having substituent groups such as alkyl groups, alkoxy groups, ester groups, nitro groups, halogen atoms, cyano groups, hydroxy groups thiol groups; or silyl groups such as an alkyl silyl group, a dialkyl silyl group, a trialkyl silyl group and a silyl group having an aryl group as a substituent.

Preferably, R67-R84 are, respectively, represent a hydrogen atom or an alkyl group, and more preferably represent a hydrogen atom.

In addition, in Formula (23), R67-R70 may form a cyclic bonding structure between any of them, at least one of R67-R70 must be bonded directly or indirectly to an acid decomposable group of the present invention, or form at least a part of the acid decomposable group.

In addition, in Formula (24), R71-R76 may form a cyclic bonding structure between any of them, at least one of R71-R76 must be bonded directly or indirectly to an acid decomposable group of the present invention, or form at least a part of the acid decomposable group.

In addition, in Formula (25), R77-R80 may form a cyclic bonding structure between any of them, at least one of R77-R80 must be bonded directly or indirectly to an acid decomposable group of the present invention, or form at least a part of the acid decomposable group.

In addition, in Formula (26), R81-R83 may form a cyclic bonding structure between any of them, at least one of R81-R83 must be bonded directly or indirectly to an acid decomposable group of the present invention, or form at least a part of the acid decomposable group

In Formula (23), X7 represents a methylene group which may have substituent groups, an ethylene group which may have substituent groups, an oxygen atom or a sulfur atom. X7 represents preferably a methylene group which may have substituent groups or an oxygen atom, and most preferably an oxygen atom.

In Formula (27), R85 represents an alkyl group which may have substituent groups, an aryl group which may have substituent groups, an alkoxy group which may have substituent groups, a thioalkoxy group which may have substituent groups, a vinyl group which may have substituent groups. R86 represents an aryl group which may have substituent groups, and preferably represents phenyl group which may have substituent groups.

In addition, R84-R86 may form a cyclic bonding structure between any of them, at least one of R84-R86 must be bonded directly or indirectly to an acid decomposable group of the present invention, or form at least a part of the acid decomposable group.

In Formula (28), R87-R90, respectively, a represent linear, branched or cyclic alkyl group having 1 to 6 carbons which may have substituent groups,

In Formula (28), R91 represents a polyvalent organic group which may have substituent groups, and preferably represents an ethylene group or propylene group.

In addition, R87-R91 may form a cyclic bonding structure between any of them, at least one of R87-R91 must be bonded directly or indirectly to an acid decomposable group of the present invention, or form at least a part of the acid decomposable group.

In the present invention, the acid decomposable group may include a part of thermally-bond-formable group or whole thermally-bond-formable group.

More preferable example of thermally-bond-formable groups for the examples of acid decomposable groups shown in the Formula (2) to (8) will be shown in the following Formula (9) and (10).

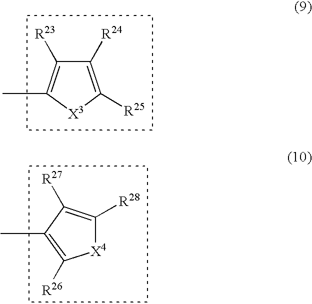

In Formula (9) and (10), X3 and X4, respectively, represent an oxygen atom, a methylene group or an ethylene group.

R23-R28, respectively, represent a hydrogen atom; a halogen atom; a hydroxy group; a thiol group; a cyano group; a nitro group; an amino group; an alkylamino group; a dialkylamino group; a linear, branched or cyclic alkyl group having 1 to 6 carbons which may have substituent groups; an aryl group having 5 to 10 carbons which may have substituent groups; an alkoxy group which may have substituent groups, thioalkoxy group which may have substituent groups; a vinyl group which may have substituent groups; a saturated or unsaturated heterocyclic group which may have substituent groups; and a silyl group which may have substituent groups. In Formula (9), R23-R25 may form a cyclic structure between any of them, and R23-R25 may form a cyclic structure with any atom groups positioning between a structure corresponding to Formula (9) and an acid decomposable group.

In Formula (10), R26-R28 may form a cyclic structure between any of them, and R26-R28 may form a cyclic structure with any atom groups positioning between a structure corresponding to Formula (10) and an acid decomposable group.

Preferable example of thermally-bond-formable groups include cyclic dienes with R23-R25 and R26-R28, which are shown in the following Formula (9) and (10), as a substituent group. As described above, these cyclic dienes may be a part of the acid decomposable groups. Such examples in which a cyclic diene is a part of the acid decomposable group are shown in the following Formula (39) to (42).

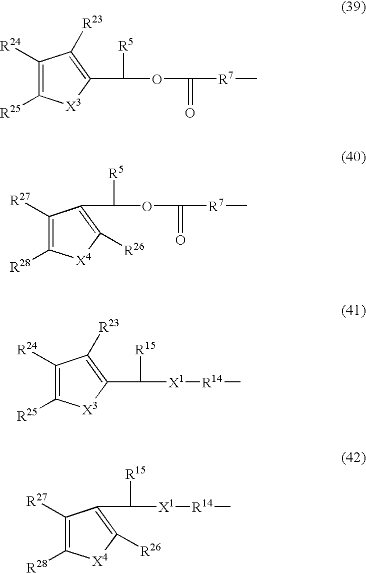

Formula (39) to (42) are corresponding to Formula (3) or (6) where R6 or R16 is shown in Formula (9) or (10) respectively. That is, in Formula (39) to (42), R5, R7, R14, R15 and X1 are respectively the same atom groups shown in the Formula (3) or (6), R23-R28, X3 and X4 are respectively the same atom groups in the Formula (9) or (10). Because a conjugated diene, especially an aromatic group such as a furan can stabilize its cationic structure generated from the acid decomposable group by a acid catalyzed decomposition reaction, in the Formula (39) to (42), the conjugated diene structure is making up a part of the acid decomposable group.

As described above, because a present invented compound have both an acid decomposable group and a thermally-bond-formable group, the present invented compound is subjected to structure changing like crosslinking or else by heating as well as subjected to decomposition by an acid treatment where the compound becomes small pieces or changes its polarity which makes it easy to be dissolved in a solvent and to be disposed of. In addition, in the course of the invention, the inventor found that an acid decomposable compound of the present invention having 1-(2-furyl)ethyloxycarbonyl groups, which shown in the formula below, possesses unique thermal property that allow the compound be stable at a certain, relatively high temperature, but be dramatically unstable above the temperature.

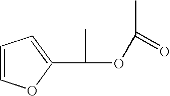

In the case of poly(1-(2-furyl)ethylmethacrylate), in the course of TGA measurement, under the condition that N₂ atmosphere, temperature rising speed was 10.0° C./min, it was completely stable under 232° C., but it was rapidly decomposed up to the temperature of 239° C. to the 60% amount. From this fact, these compounds having 1-(2-furyl)ethyloxycarbonyl groups in its molecule are also useful as termal decomposable compounds.

Moreover, the compound is a possible resist material which will exhibit excellent properties. For example, a phenolic hydroxyl group protected by t-butyl group is subjected to decomposition reaction to form the phenolic hydroxyl group and the butene. In this case, a formation of the phenolic hydroxyl group having a large polarity from the t-butyl phenyl ether group having a low polarity makes a solubility to an alkali solution higher and a solubility to a low-polar solution lower.

An acid decomposable group like the t-butyl phenyl ether group is commonly used as a photoresist resin, so that a compound of the present invention is available for a photoresist application. A polyester made up by tertiary ester bonding in its main chains is an example of a case in which a change in molecular weight cause a change in solubility.

Usually, in a photoresist application, reactions which are based on an acid catalyzed reaction caused by light irradiation cause decomposition of an acid decomposable groups, making a solubility of the light irradiated region to an alkali solution higher. On the contrary, no reactions occur in a non-light irradiated region.

The acid decomposable compound of the present invention, however, makes it possible to cause reactions which reduce a solubility to a alkali solution such as crosslinking reaction in a non-light emitted region when the compound is used as a photoresist and irradiated with light. In this case, a posi-type photoresist composition can be obtained by applying the compounds of the present invention.

From forgoing explanation, some of the most preferable compound structures of the present invention will be explained now.

One of the most preferable structures of the acid decomposable compounds of the present invention is shown in the following Formula (11) or (12).

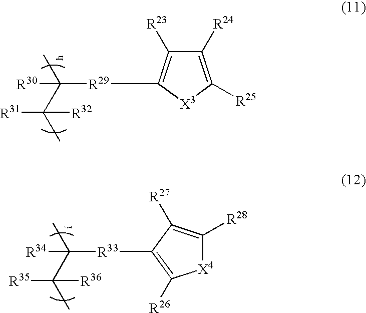

In the Formula (11) and (12), X3, X4, or R23-R28 represents the same atom group described in the Formula (9) and (10). R29 or R33 respectively represents the acid decomposable groups having the same structure as the structure of the Formula (2) to (8). “h” and “i”, respectively, represent an integer.

In Formula (11) and (12), R30-R32 and R34-36 respectively represent a hydrogen atom, a linear, branched or cyclic alkyl group having 1 to 6 carbons which may have substituent groups, aryl group having 5 to 10 carbons which may have substituent groups, alkoxy group which may have substituent groups. Also in Formula (11), R30-R32 may form cyclic bonding structure between them. A part of atom group in R29 and at a closer site to the R30 than the acid dissociative bond in R29 may form a cyclic structure with R30-32.

Also in Formula (12), R34-R36 may form a cyclic bonding structure between them. A part of atom group in R29 and at a closer site to the R34 than the acid dissociative bond in R34 may form a cyclic structure with R34-36.

Another example of the most preferable structures of the acid decomposable compounds of the present invention is shown in the following Formula (13).

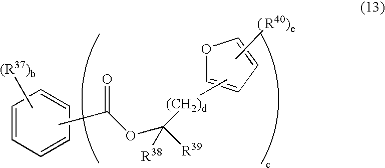

In Formula (13), R37, R38 or R40 respectively represents a hydrogen atom; a halogen atom; a hydroxy group; a thiol group; a cyano group; a nitro group; an amino group; an alkylamino group; a dialkylamino group; a linear, branched or cyclic alkyl group having 1 to 6 carbons which may have substituent groups; an aryl group having 5 to 10 carbons which may have substituent groups; an alkoxy group which may have substituent groups; a thioalkoxy group which may have substituent groups, a vinyl group which may have substituent groups, a saturated or unsaturated heterocyclic group which may have substituent groups; and a silyl group which may have substituent groups; “d” represents integer from 0 to 10; R39 represents a linear, branched or cyclic alkyl group having 1 to 6 carbons which may have substituent groups; an aryl group having 5 to 10 carbons which may have substituent groups; an alkoxy group which may have substituent groups; a thioalkoxy group which may have substituent groups; a vinyl group which may have substituent groups; a saturated or unsaturated heterocyclic group which may have substituent groups; and a silyl group which may have substituent groups, also R39 may represent hydrogen atom when d=0; “c” represents an integer from 1 to 6, “b” represents an integer from 0 to (6-c), and “e” represents an integer from 0 to 3; when “b” is 2 or more, plural R37s may be different from each other; when “e” is 2 or more, plural R40s may be different from each other; when “c” is 2 or more, the plural atom groups shown in the parenthesis relating to “c” may be different from each other.

Preferable examples of the acid decomposable compound of the present invention shown in Formula (13) are trimesic acid derivatives. Such trimesic acid derivatives can be synthesized, for example, by reacting trimesic chloride and furyl-2-methylalchol or 2-furyl-2-methylalkoxide.

Another example of the most preferable structures of the acid decomposable compounds of the present invention is shown in the following Formula (14) or (15).

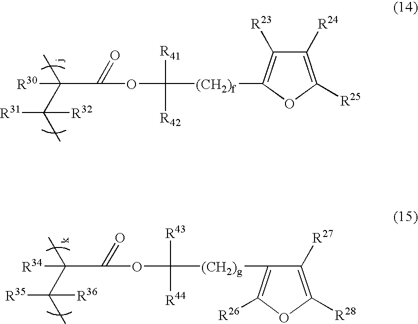

In the Formula (14) and (15), R23-R28, R30-R32 or R34-36 respectively represents the same atom group described in the Formula (11) and (12). “j” and “k”, respectively, represent an integer.

R41 and R43, respectively, represent a linear, branched or cyclic alkyl group having 1 to 6 carbons which may have substituent groups; an aryl group having 5 to 10 carbons which may have substituent groups; an alkoxy group which may have substituent groups; an thioalkoxy group which may have substituent groups; a vinyl group which may have substituent groups; a saturated or unsaturated heterocyclic group which may have substituent groups; and a silyl group which may have substituent groups.

“f” and “g” respectively represent an integer less than 10. R42 represents a linear, branched or cyclic alkyl group having 1 to 6 carbons which may have substituent groups; an aryl group having 5 to 10 carbons which may have substituent groups; an alkoxy group which may have substituent groups; a thioalkoxy group which may have substituent groups; a vinyl group which may have substituent groups; a saturated or unsaturated heterocyclic group which may have substituent groups; and a silyl group which may have substituent groups; and when “f” is 0, R42 also may represents a hydrogen atom.

R44 represents a linear, branched or cyclic alkyl group having 1 to 6 carbons which may have substituent groups; an aryl group having 5 to 10 carbons which may have substituent groups; an alkoxy group which may have substituent groups; a thioalkoxy group which may have substituent groups; a vinyl group which may have substituent groups; a saturated or unsaturated heterocyclic group which may have substituent groups; and a silyl group which may have substituent groups, and when “g” is 0, R42 also may represents a hydrogen atom.

Preferable example of the compound represented by Formula (14) or (15) is an acrylic acid or methacrylic acid derivatives. That is, it is preferable that, in Formula (14), R30=hydrogen atom or methyl group, R31 and R32=hydrogen atom, R41=hydrogen atom or alkyl group having 1 to 6 carbon atoms, R42=alkyl group having 1 to 6 carbon atoms, f is an integer from 0 to 10, R23 and R24 and R25=hydrogen atom, or in Formula (15), R34=hydrogen atom or methyl group, R35 and R36=hydrogen atom, R43=alkyl group having 1 to 6 carbon atoms, R44=alkyl group having 1 to 6 carbon atoms, g is an integer from 0 to 10, R26 and R27 and R28=hydrogen atom. In Formula (14), the case in which R30=hydrogen atom or methyl group, R31 and R32=hydrogen atom, R41=methyl group, R42=hydrogen group, f=0, R23 and R24 and R25=hydrogen atom is more preferable. In Formula (15), the case in which R34=hydrogen atom or methyl group, R35 and R36=hydrogen atom, R43=methyl group, R44=hydrogen group, g=0, R26 and R27 and R28=hydrogen atom is more preferable.

Preferable examples of the monomer units of the acid decomposable compound of the present invention shown in Formula (14) or (15) can be synthesized, for example, by reacting acryloyl chloride or methacryloyl chloride with furyl-2-methylalchol or 2-furyl-2-methylalkoxide, or acrylic acid or methacrylic acid with furyl-2-methylalchol or furyl-3-methylalchol in the presence of an acid catalyst.

### EXAMPLES

Hereinafter, the present invention will be explained further by the following practical examples, but no limitation is imposed by the examples.

Synthesis of 1-(2-furyl)ethylmethacrylate

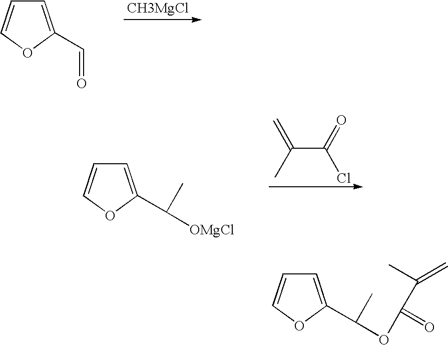

200 ml of Dried tetrahydrofuran was poured into 1-liter flask in which the atmosphere replaced with nitrogen, then 138 ml (0.414 mol) of 3M methylmagnesium chloride in tetrahydrofuran was added and stirred by a magnetic stirrer. While the flask was being cooled with ice, 35.8 g of furfural was dropped into the flask for 1 hour, then stirred for another 30 min. 0.42 ml (0.430 mol) of methacryloyl chloride was added by dropping for 1 hour, then the reaction mixture was stirred for 16 hours. 100 ml of water was added to the reaction mixture and stirred for 10 min, then the resultant water layer was removed by separating funnel. The obtained organic layer was washed with 100 ml of 5.6% NH₃ aqueous solution. Next, 200 ml of diethylether was added to the organic layer, then the organic layer was washed 3 times with water. The resultant organic layer was dried with MgSO₄, then concentrated by evaporating the solvent. The concentrated reaction mixture was diluted under reduced pressure (40 Pa). 66.89 g of 1-(2-furyl)ethylmethacrylate was obtained. Yield 72.3%, NMR spectra of it is shown in FIG. 1.

Synthesis of poly(1-(2-furyl)ethylmethacrylate)

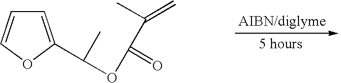

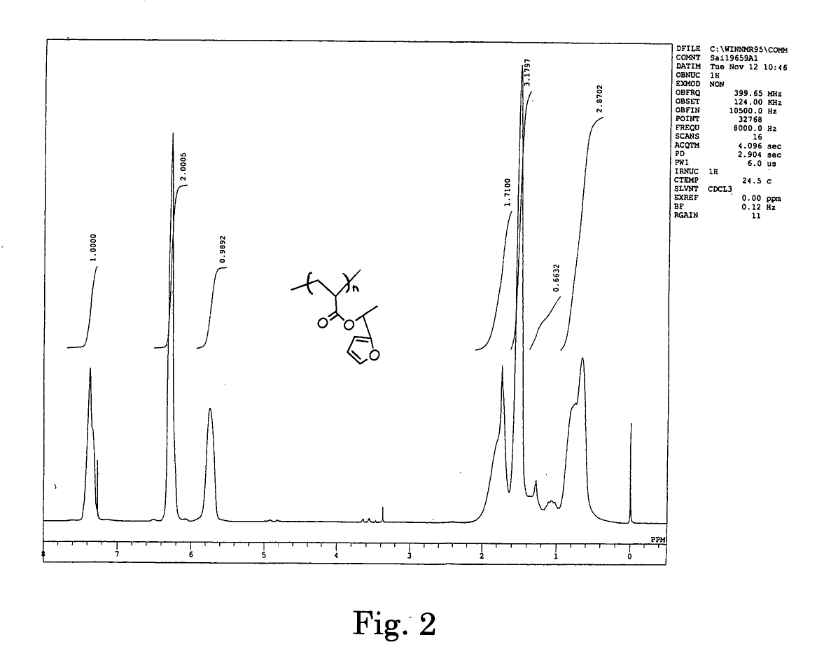

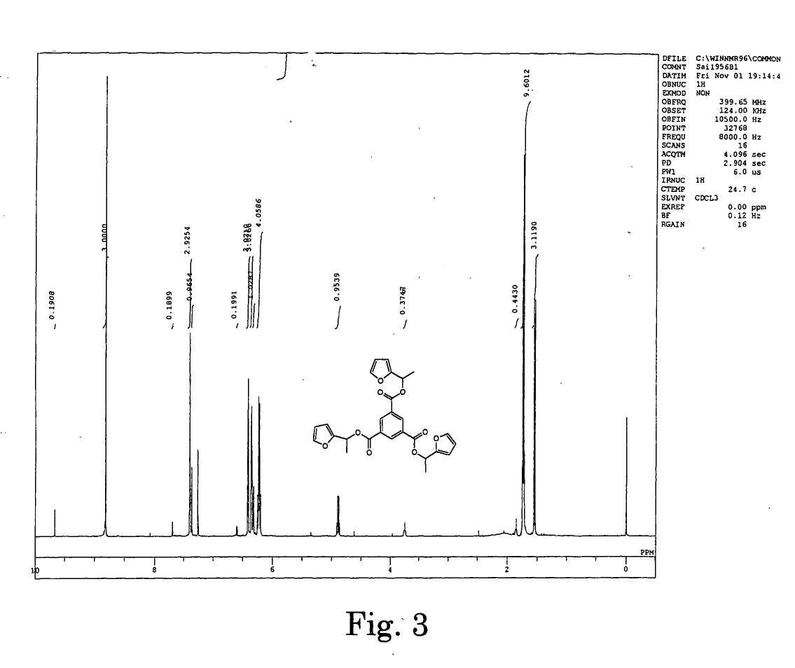

28.43 g (0.158 mol) of 1-(2-furyl)ethylmethacrylate and 1.0 g (0.0061 mol) of 2,2′-azobis(isobutyronitrile) were added into 56 ml of diglyme. The mixture was poured into a 500 ml pressure-proof tube, after the tube was closed, the tube was cooled in liquid nitrogen, then by using a vacuum pump, the pressure of the inside of the tube was reduced in order to degas. After 3 times of the degassing, the tube was placed in a oil bath (80° C.) and stirred inside for 5 hours. The reaction mixture was dropped into 2500 ml of methanol for 1 hour, white solid was precipitated. The white solid was collected by a filtration, then dried for 16 hours at 20° C. Poly(1-(2-furyl)ethylmethacrylate) was obtained in a form of white powder. Yield 92% (26.24 g), NMR chart is shown in FIG. 2.

Synthesis of 1,3,5-tris(methylurfuryloxycarbonyl)benzene

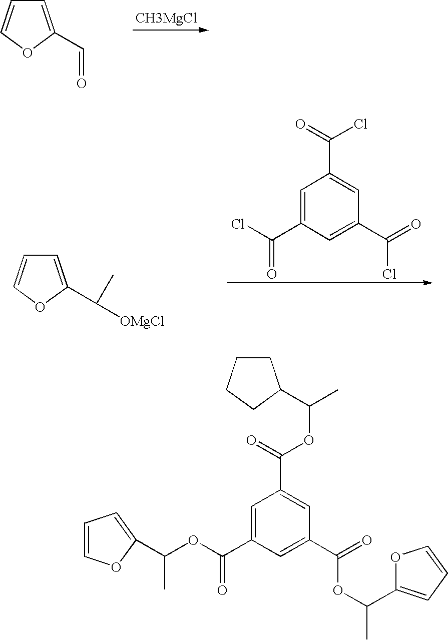

Into a flask which was nitrogen-replaced inside, 110 ml of 1.5M magnesium chloride in tetrahydrofuran was poured. While the flask was being cooled with ice bath and the solution was being stirred, 35.6 g of furfural was added to the solution by dropping for 80 min. After the dropping was finished, the solution was stirred for another 10 min. Next, 20.63 g of trimesic chloride in 60 ml of tetrahydrofuran was dropped into the solution for 80 min. 200 ml of diethylether and 200 ml of 4M ammonium chloride water solution were added to the reaction mixture and stirred. The resultant organic layer was separated, then dried with MgSO₄, then evaporated under reduced pressure. The concentrated material contained 1,3,5-tris(methylurfuryloxycarbonyl)benzene and methylfurfurylalchol in the ratio of 3:1. NMR chart of the material was shown in FIG. 3.

Acid Decomposability of 1-(2-furyl)ethylmethacrylate

021 g (0.0011 mol) of 1-(2-furyl)ethylmethacrylate and 0.1 g of p-methoxyphenol is dissolved into 2.0 g of CDCl₃, then mixed with 1.0 g of 10% H₂SO₄ in D₂O. After stirring for 12 hours at 60° C., the CDCl₃ layer was measured with NMR. The 1-(2-furyl)ethylmethacrylate was reduced to ⅓ equivalent amounts to the amounts before reaction, and methacrylic acid was generated in ⅔ equivalent amounts to the amounts of the originally added 1-(2-furyl)ethylmethacrylate. In the comparative example, which will be explained later, 2-methyl tricyclo[5.2.1.0^{2.6}] dodecanyl methacrylate-2-yl, which is a possible compound to be available as an acid decomposable component in an ArF photoresist, was not degraded under the same condition. These fact strongly, clearly shows that the poly(1-(2-furyl)ethylmethacrylate) can act as acid decomposable component in a photoresist composition.

### COMPARATIVE EXAMPLE

Acid Decomposability of Furfuryl Methacrylate

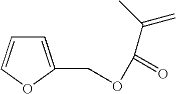

0.20 g (0.0012 mol) of furfuryl methacrylate and p-methoxyphenol were dissolved into 2.0 g of CDCl₃, then mixed with 1.0 g of 10% H₂SO₄ in D₂O. After stirring for 12 hours at 60° C., the CDCl₃ layer was measured with NMR. However, no decomposition of the furfuryl methacrylate was observed.

Acid Decomposability of 2-methyl tricyclo[5.2.1.0^{2.6}] dodecanyl methacrylate-2-yl

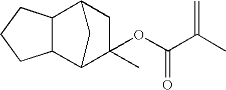

0.25 g of 2-methyl tricyclo[5.2.1.0^{2.6}] dodecanyl methacrylate-2-yl, which is reported to possess acid decomposability, and p-methoxyphenol were dissolved into 2.0 g of CDCl₃, then mixed with 1.0 g of 10% H₂SO₄ in D₂O. After stirring for 12 hours at 60° C., the CDCl₃ layer was measured with NMR. However, no decomposition of 2-methyl tricyclo[5.2.1.0^{2.6}] dodecanyl methacrylate-2-yl was observed.

What is claimed is:

1. An acid decomposable compound containing at least a core structure, acid decomposable groups which are binding to the core structure and thermally-bond-formable groups which are binding to the acid decomposable groups as shown in following Formula (1): 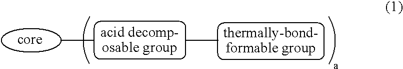
wherein the core structure represents a carbon atom, a silicon atom, an oxygen atom, a sulfur atom, a boron atom and an atom whose electron negativity difference to carbon is 2.0 or less, or represents an organic group, in addition, the core may make up all or part of the acid decomposable groups, and the acid decomposable groups represents organic groups containing at least one of the groups selected from the following groups: a tertiary ester bond, a secondary arylester bond, a tertiary oxycarbonyloxy bond, a thioester bond, an acetal bond, a tertiary ether bond, a secondary arylester bond, a tertiary thioether bond, a secondary thioether bond and these bonds partially substituted with electron donating groups, in addition, the acid decomposable groups may make up all or part of the thermally-bond-formable groups, and the thermally-bond-formable groups represents organic groups which can undergo Diels-Alder Reactions, metathesis reactions or other thermal-addition reactions, where “a” represents positive integer, and when “a” is two or more, acid decomposable groups may be different from each other and thermally-bond-formable groups may be different from each other.

2. The acid decomposable compound according to claim 1, wherein the thermally-bond-formable groups containing at least one group selected from a furan structure, maleimide structure, anthracene structure, cyclopentadiene structure, and butadiene structure.

3. The acid decomposable compound according to claim 1, wherein the core has a structure of which the Formula weight is 1000 or more.

4. The acid decomposable compound according to claim 1, wherein the core contains at least one of the units selected from an acrylic acid derivative unit, methacrylic acid derivative unit, hydroxystyrene derivative unit, norbornene derivative unit, maleic acid derivative unit and maleimide derivative unit.

5. The acid decomposable compound according to claim 1, wherein the acid decomposable group is an atom group as shown in following Formula (2) to Formula (8) and the thermally-bond-formable group is an atom group as shown in following Formula (9) or Formula (10): 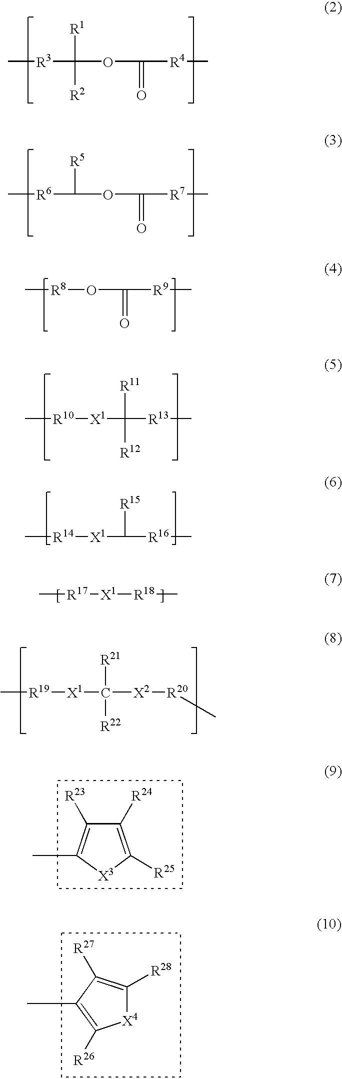
wherein Formula (2) to (8), R1, R2, R5, R1, R12, R15, R21 and R22, respectively, represent a linear, branched or cyclic alkyl group having 1 to 6 carbons which may have substituent groups; an aryl group having 5 to 10 carbons which may have substituent groups; an alkoxy group which may have substituent groups; a thioalkoxy group which may have substituent groups; a vinyl group which may have substituent groups; a saturated or unsaturated heterocyclic group which may have substituent groups; and a silyl group which may have substituent groups; in addition, R3, R4, R7, R9, R13, R19 and R20, respectively, represent a bivalent organic group which is a group selected from an alkyrene group which may have substituent groups, a vinylene group which may have substituent groups, a carbonyl group, an arylene group which may have substituent groups; a silyrene group; an oxygen atom; a sulfur atom; a secondary or tertiary boron atom; a secondary or tertiary nitrogen atom; or any combination of these said groups or atoms; in addition, in Formula (2), R1, R2, R3 and an R3 binding group (not shown, it is a part of the core structure or thermally-bond-formable group) may form a cyclic bonding structure between any of them; R4 and an R4 binding group (not shown, it is a part of the core or thermally-bond-formable group) may form a cyclic bonding structure between them; in addition, in Formula (3) and (6), R6 and R16, respectively, represent an organic group which containing at least the following group at the closer side to the acid dissociative site of it: an arylene group which may have substituent groups, a vinylene group which may have substituent groups, a methylene group which have electron donating groups, a vinyleneoxy group which may have substituent groups, or a silyrene group which may have substituent groups; in addition, in Formula (3), R5, R6 and an R6 binding group (not shown, it is a part of the core or thermally-bond-formable group) may form a cyclic bonding structure between any of them; R7 and an R7 binding group (not shown, it is a part of the core or thermally-bond-formable group) may form a cyclic bonding structure between them; in addition, in Formula (4) and (7), R8 and R18 respectively represent an organic group which containing at least the following group at the closer side to the acid dissociative site of it: a bivalent organic group selected from a tetrahydropiranylene; an arylene having electron donating group; a vinylene group having electron donating groups; an aryloxymethylene group which may have substituent groups; an N-phthalimide methylene group which may have substituent groups; a 9-anthryl methylene group which may have substituent groups; and silylene groups which may have substituent groups; in addition, in Formula (4), R8 and an R8 binding group (not shown, it is a part of the core or thermally-bond-formable group) may form a cyclic bonding structure between them, R9 and an R9 binding group (not shown, it is a part of the core or thermally-bond-formable group) may form a cyclic bonding structure between them; in addition, in Formula (5) to (8), X1 and X2 respectively represent oxygen atom or sulfur group; in addition, in Formula (5) to (7), R10, R14 and R17 respectively represent an organic group which containing at least the following group at the closer side to an acid dissociative site of it: a bivalent arylene group which may have substituent groups and a bivalent vinylene group which conjugates to an aryl group and may have substituent groups; also in Formula (5), R11, R12, R13 and an R13 binding group (not shown, it is a part of the core or thermally-bond-formable group) may form a cyclic bonding structure between any of them, R10 and an R10 binding group (not shown, it is a part of the core or thermally-bond-formable group) may form a cyclic bonding structure between them; also in Formula (6), R15, R16 and an R16 binding group (not shown, it is a part of the core or thermally-bond-formable group) may form a cyclic bonding structure between any of them, R14 and an R14 binding group (not shown, it is a part of the core or thermally-bond-formable group) may form a cyclic bonding structure between them; also in Formula (7), R17 and an R17 binding group (not shown, it is a part of the core or thermally-bond-formable group) may form a cyclic bonding structure between them, R18 and an R18 binding group (not shown, it is a part of the core or thermally-bond-formable group) may form a cyclic bonding structure between them; also in Formula (8), R21, R22, R19 and an R19 binding group (not shown, it is a part of the core or thermally-bond-formable group) may form a cyclic bonding structure between any of them, R21, R22, R20 and an R20 binding group (not shown, it is a part of the core or thermally-bond-formable group) may form a cyclic bonding structure between any of them, but when R21 forms a cyclic bonding structure with R19 or the R19 binding group, R21 or R22 does not form a cyclic bonding structure with R20 or the R20 binding group; when R21 forms a cyclic bonding structure with R20 or the R20 binding group, R21 or R22 does not form a cyclic bonding structure with R19 or the R19 binding group; when R22 forms a cyclic bonding structure with R19 or the R19 binding group, R22 does not form a cyclic bonding structure with R20 or the R20 binding group; in addition, in Formula (9) and (10), X3 and X4, respectively, represent an oxygen atom, a methylene group or an ethylene group; in addition, R23-R28, respectively, represent a hydrogen atom; a halogen atom; a hydroxy group; a thiol group; a cyano group; a nitro group; an amino group; an alkylamino group; a dialkylamino group; a linear, branched or cyclic alkyl group having 1 to 6 carbons which may have substituent groups; an aryl group having 5 to 10 carbons which may have substituent groups; an alkoxy group which may have substituent groups, thioalkoxy group which may have substituent groups; a vinyl group which may have substituent groups; a saturated or unsaturated heterocyclic group which may have substituent groups; and a silyl group which may have substituent groups. In Formula (9), R23-R25 may form a cyclic structure between any of them, and R23-R25 may form a cyclic structure with any atom groups positioning between a structure corresponding to Formula (9) and an acid decomposable group.

6. The acid decomposable compound according to claim 5, wherein the acid decomposable compound contains a structure unit shown in the following Formula (11) or (12): 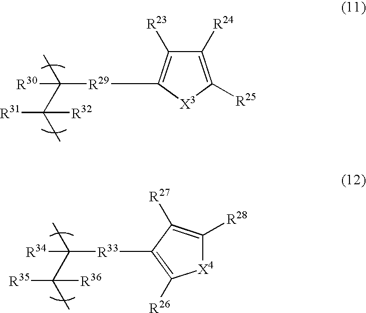
wherein Formula (11) and (12), X3, X4 and R23-R28 represent the same atom groups as described in claim 5; R29 and R33, respectively, represent the groups described in claim 5 as Formula (2) to Formula (8); “h” and “i”, respectively, represent an integer; R30-R32 and R34-36, respectively, represent a hydrogen atom, a linear, branched or cyclic alkyl group having 1 to 6 carbons which may have substituent groups, aryl group having 5 to 10 carbons which may have substituent groups, alkoxy group which may have substituent groups; in addition, in Formula (11), R30-R32 may form a cyclic structure between any of them, and R30-R32 may form a cyclic structure with any atom positioned between a structure corresponding to an acid decomposable bond in R29 and R30-R32; in Formula (12), R34-R36 may form cyclic structure between any of them, and R34-R36 may form a cyclic structure with any atom positioned between a structure corresponding to an acid decomposable bond in R33 and R34-R36.

7. The acid decomposable compound according to claim 5, wherein the acid decomposable compound contains a structure shown in following Formula (13): 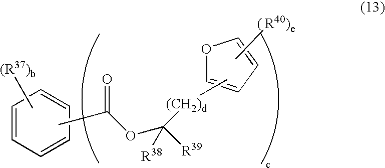
wherein R37, R38 or R40 respectively represents a hydrogen atom; a halogen atom; a hydroxy group; a thiol group; a cyano group; a nitro group; an amino group; an alkylamino group; a dialkylamino group; a linear, branched or cyclic alkyl group having 1 to 6 carbons which may have substituent groups; an aryl group having 5 to 10 carbons which may have substituent groups; an alkoxy group which may have substituent groups; a thioalkoxy group which may have substituent groups, a vinyl group which may have substituent groups, a saturated or unsaturated heterocyclic group which may have substituent groups; and a silyl group which may have substituent groups; “d” represents integer from 0 to 10; R39 represents a linear, branched or cyclic alkyl group having 1 to 6 carbons which may have substituent groups; an aryl group having 5 to 10 carbons which may have substituent groups; an alkoxy group which may have substituent groups; a thioalkoxy group which may have substituent groups; a vinyl group which may have substituent groups; a saturated or unsaturated heterocyclic group which may have substituent groups; and a silyl group which may have substituent groups, also R39 may represent hydrogen atom when d=0; “c” represents an integer from 1 to 6, “b” represents an integer from 0 to (6-c), and “e” represents an integer from 0 to 3; when “b” is 2 or more, plural R37s may be different from each other; when “e” is 2 or more, plural R40s may be different from each other; when “c” is 2 or more, the plural atom groups shown in the parenthesis relating to “c” may be different from each other.

8. The acid decomposable compound according to claim 6, wherein the acid decomposable compound contains a structure unit shown in following Formula (14) or (15): 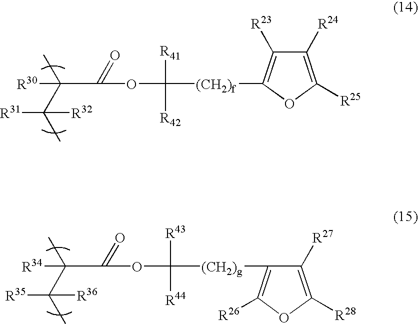
wherein R23-R28, R30-R32 and R34-R36 represent the same atom groups as described in the claim 6; “j” and “k”, respectively, represent an integer; R41 and R43, respectively, represent a linear, branched or cyclic alkyl group having 1 to 6 carbons which may have substituent groups; an aryl group having 5 to 10 carbons which may have substituent groups; an alkoxy group which may have substituent groups; an thioalkoxy group which may have substituent groups; a vinyl group which may have substituent groups; a saturated or unsaturated heterocyclic group which may have substituent groups; and a silyl group which may have substituent groups; “f” and “g” respectively represent an integer less than 10. R42 represents a linear, branched or cyclic alkyl group having 1 to 6 carbons which may have substituent groups; an aryl group having 5 to 10 carbons which may have substituent groups; an alkoxy group which may have substituent groups; a thioalkoxy group which may have substituent groups; a vinyl group which may have substituent groups; a saturated or unsaturated heterocyclic group which may have substituent groups; and a silyl group which may have substituent groups; and when “f” is 0, R42 also may represents a hydrogen atom; R44 represents a linear, branched or cyclic alkyl group having 1 to 6 carbons which may have substituent groups; an aryl group having 5 to 10 carbons which may have substituent groups; an alkoxy group which may have substituent groups; a thioalkoxy group which may have substituent groups; a vinyl group which may have substituent groups; a saturated or unsaturated heterocyclic group which may have substituent groups; and a silyl group which may have substituent groups, and when “g” is 0, R42 also may represents a hydrogen atom.
